SEQUENCE LISTING (12 sequences)

SEQ ID NO: 1 - DNA, 20 bp, Artificial
  Primer PR30
ctcgtacctttagtcggatg

SEQ ID NO: 2 - DNA, 22 bp, Artificial
  Primer PR31
ggtagctttgattacatagccg

SEQ ID NO: 3 - DNA, 22 bp, Artificial
  Primer PR32
gatggtcgtctgttatcgactc

SEQ ID NO: 4 - DNA, 29 bp, Artificial
  Primer PR33
gtctgatagtgtattagcagatgtaaaac

SEQ ID NO: 5 - DNA, 23 bp, Artificial
  Primer PR43
gactacacaaatcagcgatttcc

SEQ ID NO: 6 - DNA, 17 bp, Artificial
  Primer PR44
cttctgacctgcggtcg

SEQ ID NO: 7 - DNA, 34 bp, Artificial
  Primer PRF1
ggccgcggccgccactagatgaacatgacaccgg

SEQ ID NO: 8 - DNA, 32 bp, Artificial
  Primer PRF2
ggccccccggggcattacgtgttgtttgttgc

SEQ ID NO: 9 - DNA, 32 bp, Artificial
  Primer PRF3
ggcccctgcaggcaacaaacaacacgtaatgc

SEQ ID NO: 10 - DNA, 33 bp, Artificial
  Primer PRF4
cgcccgtcgaccttctttagaggaaatcgctgc

SEQ ID NO: 11 - DNA, 32 bp, Artificial
  Primer PRF5
ggccctacgtagcaacaaacaacacgtaatgc

SEQ ID NO: 12 - DNA, 40 bp, Artificial
  Primer PRF6
ggccgcggccgcctctatgtttttgtagatatctttttcc

(19) United States
(12) Patent Application Publication
(10) Pub. No.: US 2005/0287162 A1
(43) Pub. Date: Dec. 29, 2005

(54) Recombinant fowlpox virus

(76) Inventors: Robert Baier, Oberschleissheim (GB); Denise Boulanger, Southampton (GB); Volker Erfle, Muenchen (DE); Gerd Sutter, Muenchen (DE)

Correspondence Address: JENKINS, WILSON & TAYLOR, P. A., 3100 TOWER BLVD, SUITE 1400, DURHAM, NC, 27707, US

(21) Appl. No.: 10/514,056

(22) PCT Filed: May 13, 2003
(86) PCT No.: PCT/EP03/04991

§ 371 Date: Jul. 8, 2005

(51) Int. Cl. A61K 39/29; C12N 15/863; C12N 7/00; C12N 5/06

(52) U.S. Cl. 424/189.1; 435/456; 435/349; 435/235.1; 435/252.33

(57) ABSTRACT

The invention relates to a recombinant fowlpox virus (FWPV) and a DNA vector containing gene sequences for one such recombinant fowlpox virus. The invention also relates to a pharmaceutical composition containing said recombinant fowlpox virus or a DNA vector, to the use of said recombinant fowlpox virus for treating infectious diseases or tumour diseases, and to a method for producing said recombinant fowlpox virus or DNA vector. The invention further relates to eukaryote cells or prokaryote cells containing the recombinant DNA vector or the recombinant fowlpox virus. The invention is based on the identification of the FWPV-F11L gene as a novel insertion site for foreign DNA.

The present invention relates to a recombinant fowlpox virus (FWPV) as well as to a DNA vector containing gene sequences for such a recombinant fowlpox virus. Furthermore the invention pertains to a pharmaceutical composition comprising the recombinant fowlpox virus or a DNA vector, the use of the recombinant fowlpox virus for the treatment of infectious diseases or tumor diseases as well as to a method for the preparation of the recombinant fowlpox virus or the DNA vector. Eventually the present invention relates to eukaryotic cells or prokaryotic cells containing the recombinant DNA vector or the recombinant fowlpox virus.

Pox viruses of different genera have already been established as recombinant vaccine vectors (Moss, 1996; Paoletti, 1996). It is known from avian pox viruses including fowlpox viruses (FWPV) as a prototypic member that they replicate only in avian cells. In mammalian cells, the virus propagation is blocked at different times in the replication cycle depending on the cell type, but there is a virus-specifically controlled gene expression (Taylor et al., 1988; Somogyi et al., 1993). This property was utilized to develop recombinant candidate avian pox viruses as safe, non-replicating vectors for vaccination of mammalians including humans against infectious diseases and cancer (Wang et al., 1995; Perkus et al., 1995; Roth et al., 1996). Some of these vaccines have already been tested in clinical phase I (Cadoz et al., 1992; Marshall et al., 1999, Berencsi et al., 2001) or phase II studies (Belshe et al., 2001).

Future, more complex vaccination strategies will probably require the simultaneous expression of different antigens or the expression of a combination of antigens and immuno-co-stimulatory molecules (Leong et al., 1994; Hodge et al., 1999). These genes may be inserted either in the form of a single cassette into one site of the viral genome or may be inserted successively so that already constructed vector viruses can be continuously improved. In the latter case it is desired to have a choice among different stable insertion sites and to be able to eliminate the selectable markers so that the same selection strategy can be repeated for insertion into different sites. Furthermore, the presence of a marker gene cannot be recommended in the case of a vaccine for human use. By means of shot-gun insertion strategies several insertion sites have been identified in the FWPV genome (Taylor et al., 1988; Jenkins et al., 1991). Moreover, the insertion of foreign genes has been targeted in the viral genome into the region of the terminal inverted repeats (Boursnell et al., 1990), to non-essential gene such as the thymidine kinase gene (Boyle & Coupar, 1988) or to regions between coding gene sequences (Spehner et al., 1990).

Several strategies have been described for the generation of recombinant viruses from which the marker gene used for plaque isolation had been deleted after use.

The first strategy is the widely used method of dominant selection described by Falkner and Moss (1990) wherein the selectable marker is present within the plasmid sequence outside of the insertion cassette. Recombinant viruses generated by a single cross-over event and containing the complete plasmid sequence are obtained in the presence of selection medium. Due to the presence of the repeated sequences of the flanking regions these recombinant viruses are unstable. In the absence of selection medium, the marker gene located between these repeats is deleted after a second recombination which results either in the production of the wild-type (wt) virus or of a stable recombinant virus. The latter must again be isolated according to the plaque method and subsequently identified by means of PCR or Southern blotting.

A second method is based on the observation that in recombinant FWPV which expressed the target protein and β-galactosidase each under the control of the P7.5 promoter in direct repeat orientation a homologous recombination occurred between the promoter repeats leading to deletion of the lacZ gene (Spehner et al., 1990). For this reason, white plaques were formed by recombinant viruses which had lost the marker gene. A similar strategy has been developed to produce recombinant MVA virus using the regulatory vaccinia virus K1L gene as a transient selectable marker which is eliminated by means of intragenomic homologous recombination Staib et al., 2000).

FWPV grows more slowly than vaccinia virus. Maintaining of the full replication ability of recombinant viruses is of high importance for the generation as well the use of potential FWPV vaccination viruses.

In contrast to vectors existing to date, the present invention is based on the object to provide a recombinant fowlpox virus resulting in an increased vector stability following insertion of foreign DNA as well as a higher safety in the use as a vaccine vector and concomitantly maintaining full replication ability and optimal efficiency during the selection of recombinant viruses.

These object have been achieved by the subject matter of the independent claim. Preferred embodiments of the invention have been described in the dependent claims.

The solution according to the invention is based on the identification of the FWPV-F11L gene as a novel insertion site for foreign DNA. Viruses mutated in F11L efficiently replicate following infection of CEF (chicken embryo fibroblasts). The utility of F11L vector plasmids which allow for transient expression of the marker gene has been shown by the rapid production of recombinant FWPV viruses stably producing the tumor model antigen, tyrosinase.

The F11L gene of FWPV is already known per se and has been precisely identified. In the publication of Afonso et al. 2000, the F11L gene homologue has been precisely identified as ORF FPV110 with the genomic position 131.387-132.739. However, Afonso et al. do not disclose the property of the F11L gene as an integration site for foreign DNA.

The use of the F11L gene as an integration site for foreign DNA offers several unexpected advantages: first, it has been surprisingly found that the recombinant fowlpox viruses containing one or more insertions of foreign DNA within the F11L gene have an increased vector stability as compared to conventional vectors. Furthermore, the recombinant FWPVs according to the invention have proven to be very save in the in vivo use as vaccine vectors. Another advantage of the insertion into the F11L gene according to the invention is that the insertion may be carried out at any site of the gene.

According to a basic thought the present invention consequently provides a recombinant fowlpox virus (FWPV) which contains at least one insertion of a foreign DNA into the F11L gene. As already mentioned above, the insertion is carried out in position 131.387-132.739 of the FWPV genome. Although the insertion may basically take place at any position of the F11L gene, an insertion into the genomic region defined by nucleotide position 131.387-132.739 of the fowlpox virus genome is preferred.

In the context of the present invention, as the foreign DNA there is generally meant any DNA which is introduced into the DNA of an organism, a cell, or a virus, etc. from which it is not derived by means of genetic engineering.

According to a preferred embodiment the foreign DNA contains at least one foreign gene optionally in combination with a sequence for the regulation of the expression of the foreign gene.

The foreign gene contained in the recombinant fowlpox virus (FWPV) of the present invention encodes a polypeptide which preferably is of therapeutic use and/or encodes a detectable marker and/or a selectable gene.

Reporter gene as used herein refers to genes the gene product of which can be detected by means of simple biochemical or histochemical methods. Synonymous for the term reporter gene are indicator gene or marker gene.

In the context of the present invention, selectable gene or selectable marker, respectively, refers to genes which provide for viruses or cells, respectively, in which the respective gene products are produced a growth advantage or survival advantage, respectively, over other viruses or cells, respectively, which do not synthesize the respective gene product. Selectable markers which are preferably used are the genes for E. coli guanine phosphoribosyl transferase, E. coli Hygromycin resistance and neomycin resistance.

The foreign DNA sequence may be a gene which for example encodes a pathogenic agent or a component of a pathogenic agent, respectively. Pathogenic agents refers to viruses, bacteria and parasites which can cause a disease as well as to tumor cell which exhibit uncontrolled growth within an organism and thus can lead to pathological growth. Examples of such pathogenic agents are described in Davis, B. D. et al., (Microbiology, 3. edition, Harper International Edition). Preferred pathogenic agents are components of influenza viruses or measles or of respiratory syncytial viruses, of Dengue viruses, of Human Immunodeficiency viruses, for example HIV I and HIV II, of human hepatits viruses, for example HCV and HBV, of herpes viruses, of papilloma viruses, of the malaria Plasmodium falciparum, and of the mycobacteria causing tuberculosis.

As specific examples of components of pathogenic agents there my be e.g. mentioned envelope proteins of viruses (HIV Env, HCV E1/E2, influenza virus HA-NA, RSV F-G), regulatory virus proteins (HIV Tat-Rev-Nef, HCV NS3-NS4-NS5), the protective antigen protein of Bacillus anthracis, merozoite surface antigen, and circumsporozoite protein of Plasmodium falciparum, the tyrosinase protein as a melanoma antigen, or the HER-2/neu protein as an antigen of adenocarcinomas of humans.

Preferred genes encoding tumor-associated antigens are those which are encoded by melanoma-associated antigens, for example tyrosinase, tyrosinase-related proteins 1 and 2, of cancer-tests antigens or tumor-testes-antigens, respectively, for example MAGE-1, -2, -3, and BAGE, for non-mutated shared antigens or antigens which are shared by several tumor types, respectively, which are overexpressed on tumors, such as Her-2/neu, MUC-1 and p53.

Particularly preferred are polypeptides which are a component of HIV, Mycobacterium spp. or Plasmodium falciparum or are a component of a melanoma cell.

Components generally refers to components of those cited above which exhibit immunological properties, that means which are capable of inducing an immune reaction in mammalians, particularly in humans (e.g. surface antigens).

For the foreign DNA sequence or the gene to be able to be expressed it is necessary that regulatory sequences required for the transcription of the gene are present on the DNA. Such regulatory sequences (referred to a promoters) are known to those skilled in the art, for example a pox virus-specific promoter can be used.

Preferably the detectable marker is a beta-galactosidase, beta-glucuronidase, a luciferase, or a green-fluorescent protein.

According to a preferred embodiment the marker gene and/or selectable gene can be eliminated. As already detailed in the beginning, this property provides a great advantage because the same selection strategy can be repeated for the insertion at different sites. Furthermore, the presence of a marker gene is not to be recommended for a vaccine for human use. The deletion of these gene sequences from the genome of the final recombinant virus is carried out quasi “automatically” by means of an intragenomic homologous recombination between identical gene sequences flanking the marker selectable gene expression cassette.

According to another basic thought the present invention provides a DNA vector containing a recombinant fowlpox virus according to the invention or functional parts thereof which contain at least one insertion of a foreign DNA into the F11L gene and further preferred a replicon for the replication of the vector within a pro- or eukaryotic cell and a selectable gene or marker gene selectable in pro- or eukaryotic cells. Useful cloning and expression vectors for the use with prokaryotic and eukaryotic hosts are described in Sambrook, et al., in Molecular Cloning: A Laboratory Manual, 2^{nd} Edition, Cold Spring Harbor, N.Y. (1989).

The DNA vectors of the present invention play a role in an independent unit capable of replication which have the capability of DNA replication in suitable host cells. Thus, the foreign DNA which is not capable of replication is passively replicated as well and can afterwards be isolated and purified together with the vector. Besides the recombinant fowlpox virus gene sequences of the present invention the DNA vector can also include the following sequence elements: enhancers for enhancing the gene expression, promoters which are a prerequisite for gene expression, origins of replication, reporter genes, selectable genes, splicing signals, and packaging signals.

The DNA vector according to the invention mainly serves as a transfer vector to enable in a virus-infected cell via homologous recombination the insertion of foreign genes. Generally, it is used in the context of a fowlpox virus infection since the regulatory elements are dependent on the presence of other viral proteins.

According to the invention, the recombinant fowlpox virus or the DNA vector is provided in a pharmaceutical composition which comprises these in combination with pharmaceutically acceptable auxiliary agents and/or carriers. The pharmaceutical Composition preferably is a vaccine.

To prepare a vaccine, the FWPVs generated according to the invention are converted into a physiologically acceptable form. This may be carried out on the basis of the many years of experience in the preparation of vaccines used for the vaccination against pocks (Kaplan, Br. Med. Bull. 25, 131-135 [1969]). Typically, about 10⁶-10⁷ particles of the recombinant FWPV are lyophilized in 100 ml phosphate buffered saline (PBS) in the presence of 2% peptone and 1% human albumin in a vial, preferably in a glass vial. The lyophilisate may contain filler or diluting agents, respectively, (such as for example mannitol, dextrane, sugar, glycine, lactose or polyvinylpyrrolidone) or other auxiliary agents (for example antioxidants, stabilizers, etc.) suitable for parenteral administration. The glass vial is then closed or sealed, respectively, and can be stored preferably at temperatures of below −20° C. for several months.

For vaccination, the Lyophilisate can be dissolved in 0.1 to 0.2 ml of an aqueous solution, preferably physiological saline, and administered by the parenteral rote, for example by intradermal inoculation. The vaccine according to the invention is preferably injected by the intradermal route. A slight swelling and a rash and sometimes also an irritation can occur at the site of injection. The route of administration, the dose, and the number of administrations can be optimised by those skilled in the art in a known manner. Where applicable, it is convenient to administer the vaccine several times over al prolonged time period to achieve a high level of immune reactions against the foreign antigen.

The above-mentioned subject matters, i.e. the recombinant fowlpox virus, the DNA vector or the pharmaceutical composition are preferably used for the treatment of infectious disease or tumor diseases, as defined above.

The fowlpox virus according to the invention, the DNA vector or the pharmaceutical composition can be used either alone (e.g. as a vaccine) or in the context of a so-called prime boost approach in a prophylactic or therapeutic manner. In other words, by a repeated administration of a vaccination dose of the fowlpox virus according to the invention the immune reaction against the fowlpox virus vaccine can be further enhanced.

It is of particular advantage to combine the fowlpox viruses of the present invention with other viral vectors, for example MVA.

In the frame of a combination vaccination, as mentioned above, there may be used for example MVA or other vaccinia viruses belonging to the genus of orthopoxviruses. It is known that certain strains of vaccinia viruses have been used for many years as live vaccines for the immunization against pox, for example the Elstree strain of the Lister Institute in the United Kingdom. Vaccinia viruses have also been used often as vectors for the generation and delivery of foreign antigens (Smith et al., Biotechnology and Genetic Engineering Reviews 2, 383-407 [1984]). Vaccinia viruses are among the best examined live vectors and exhibit for example specific features which support their use as a recombinant vaccine: they are highly stable, can be prepared in a cost-effective manner, can be easily administered and are able to incorporate high amounts of foreign DNA. The vaccinia viruses have the advantage that they both induce antibody and cytotoxic reactions and enable the presentation of antigens to the immune system in a more natural way and have been successfully used as a vector vaccine for the protection against infectious diseases.

However, vaccinia viruses are infectious for humans and their use as an expression vector in the laboratory is limited by safety concern and regulations. Most of the recombinant vaccinia viruses described in the literature are based on the Western Reserve (WR) strain of vaccinia viruses. It is known, however, that this strain exhibits a high level of neurovirulence and thus is only poorly adapted for the use in man (Morita et al, Vaccine 5, 65-70 (1987)).

Safety concern with respect to the standard strains of VV have been addressed by the development of vaccinia vectors from highly attenuated virus strains characterized by their limited capability of propagation in vitro and their avirulence in vivo. Based on the Ankara strain, there has been thus cultivated the so-called modified vaccinia virus Ankara (MVA). The MVA virus was deposited according to the requirements of the Budapest treaty at CNCM on December, 15, 1987 under the deposition number I-721.

However, also other avirulent vaccinia viruses and pox virus vectors with similar properties can also be employed for the above-mentioned vaccination schedule, e.g. recombinant forms of the vaccinia viruses NYVAC, CV-I-78, LC16m0, and LC16 m8 as well as recombinant parapox viruses, such as e.g. the attenuated Orf virus D1701. Besides pox viruses, adenoviruses (particularly human adenovirus 5), orthomyxoviruses (particularly influenza viruses), herpes viruses (particularly human or equine herpes viruses, respectively), or alpha viruses (particularly Semliki Forest viruses, Sindbis viruses, and equine encephalitis viruses—VEE) may be employed as other viral vectors.

In the frame of a prime-boost approach the fowlpox vector according to the invention is preferably administered in the first immunization, i.e. the priming.

A vaccination schedule according to the invention which may be for example used in the frame of a protective vaccination against infectious diseases or tumor diseases or also in the treatment of the same is carried out as follows:

A method according to the invention for immunization of an animal, preferably a human being, preferably comprises the following steps:
a) priming of an animal with a therapeutically effective amount of a fowlpox virus according to the invention, a DNA vector or a pharmaceutical composition according to the invention
b) optionally repeating said step a) between one and three times after between one week and eight months; and
c) boosting of the animal with a therapeutically effective amount of another viral vector containing the same foreign DNA as the fowlpox vector according to the invention.

Preferably, the priming step is carried out twice prior to the boosting step, and particularly preferred the priming steps are carried out at the beginning of the treatment and in week three to five, preferably week four of the immunization, wherein the boosting step is carried out in week eleven to thirteen, preferably week twelve of the immunization.

In this respect, the present invention is also directed to a combined preparation for the successive use of the individual components mentioned above for a vaccination. Such a combined preparation consists of the following components:
a) the recombinant fowlpox virus according to the invention or the DNA vector according to the invention, optionally containing a pharmaceutically acceptable carrier,
b) another viral vector encoding the same foreign antigen as the fowlpox virus or the DNA vector according to a).

The prime-boost protocol mentioned above provides for a better immune reaction than a vaccination with either fowlpox viruses according to the present invention or another vector, such as MVA alone.

The method according to the invention for the preparation of a recombinant fowlpox virus or DNA vector comprises introducing foreign DNA into the F11L gene of a fowlpox virus by recombinant DNA techniques. Preferably the introduction is carried out by homologous recombination of the virus DNA with the foreign DNA containing F11L-specific sequences, followed by propagation and isolation of the recombinant virus or the DNA vector.

Furthermore, the present invention provides eukaryotic cell or prokaryotic cells containing the recombinant DNA vector or the recombinant FWPV according to the invention. As a prokaryotic cell there is preferably used a bacterial cell, preferably an E. coli cell. As the eukaryotic cells, there my be used avian cells, preferably chicken cells, or a cell derived from a mammal, preferably a human cell wherein human embryonic stem cells as well as human germ line cells are excluded.

The DNA vector according to the invention may be introduced into the cells for example by transfection, such as by means of calcium phosphate precipitation (Graham et al., Virol. 52, 456-467 [1973]; Wigler et al., Cell 777-785 [1979]), by menas of electroporation (Neumann et al., EMBO J. 1, 841-845 [1982]), by means of microinjection (Graessmann et al., Meth. Enzymology 101, 482-492 (1983)), by means of liposomes (Straubinger et al., Methods in Enzymology 101, 512-527 (1983)), by means of spheroblasts (Schaffner, Proc. Natl. Acad. Sci. USA 77, 2163-2167 (1980)) or by other methods which are known to those skilled in the art. Preferably, transfection by means of calcium phosphate precipitation is used.

In the following, the present invention will be illustrated by Examples and the accompanying Figures, which show:

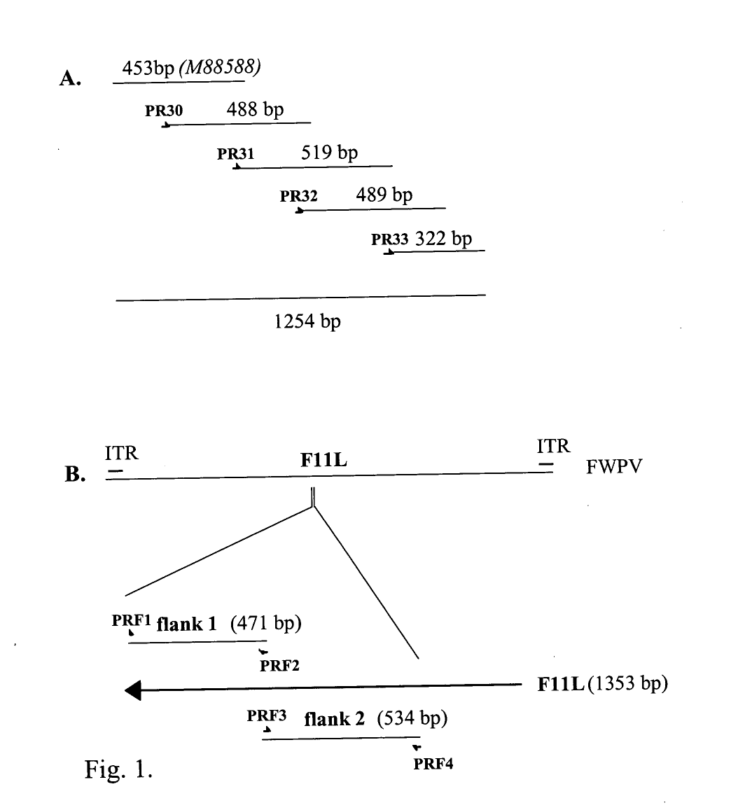

FIG. 1: (A) Primer walking sequencing strategy for the sequencing of FWPV-F11L. The length of each sequencing reaction is shown. (B) Schematic representation of the FWPV genome showing the inverted terminal repeats (ITR) and the central location of the F11L gene, as well as a representation of the preparation of F11L gene sequences which were used as flanking sequences for homologous recombination. The positions along the F11L ORF for primers F1 and F2 used for the amplification of flank 1 as well as the primers F3 and F4 used for the amplification of flank 2 are shown.

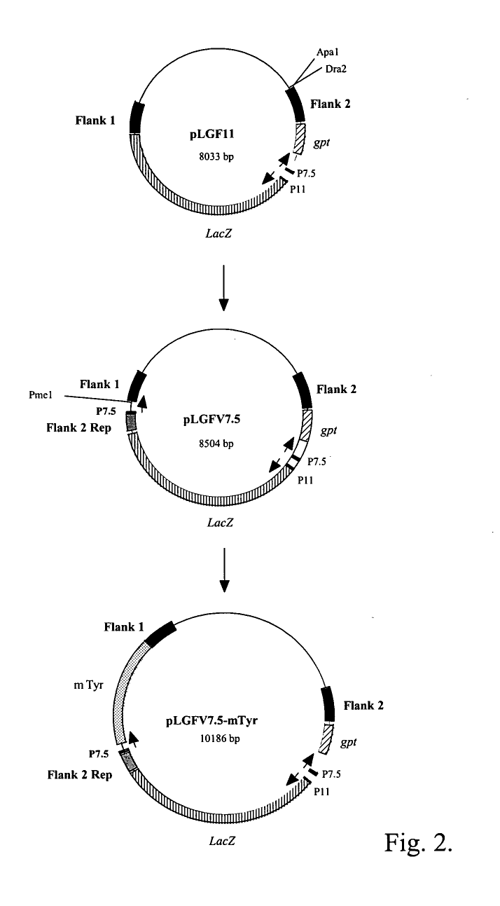

FIG. 2: Schematic maps of the insertion plasmid pLGF11 used in the preparation of viruses with mutant F11L, the vector plasmid pLGFV7.5, and of pLGFV7.5-mTyr used in the preparation of FWPV-tyrosinase recombinants. The sequences flank 1 and flank 2 derived from FWPV-F11L shown as black boxes direct the homologous recombination between the plasmid and the viral genomic DNA. The E. coli lacZ and gpt genes serve as selectable markers (shown as grey boxes). P7.5 and P11 are well characterized vaccinia virus-specific promoters the transcriptional direction of which is indicated by arrows. A unique PmeI restriction site in pLGFV7.5 can be used for insertion of foreign genes which are placed under the control of P7.5. The gene encoding tyrosinase (mTyr) from mouse serves as a first recombinant model gene.

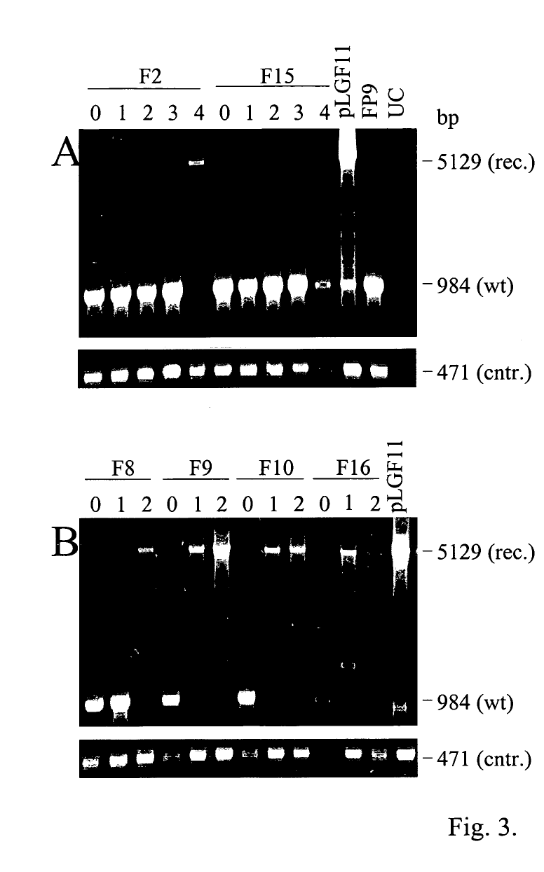

FIG. 3: PCR analysis of viral DNA from viruses with mutant F11L generated following transfection with undigested (A) or linearized pLGF11 plasmid DNA (B). The upper panels show the result of the PCR reactions using primers F1 and F4 resulting in either a band with high MG for the recombinant viruses (rec.) or in a band with a low MG for the wt virus (wt). The lower panels show the control (cntr.) PCR reactions using primers F1 and F2 showing the respective amount of viral DNA in each sample. The number of plaque purifications for each isolate is indicates starting with 0 which corresponds to the initially picked plaque isolate. pLGF11 is used as a control matrix DNA; FP9 designates the wt virus DNA control and UC is control DNA from uninfected cells.

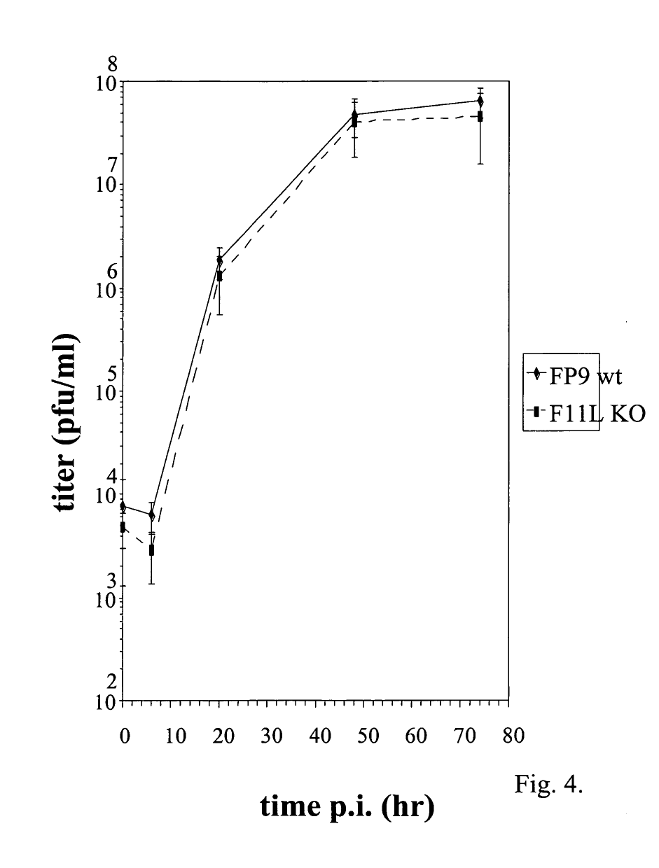

FIG. 4: Multistep growth curve experiment. CEF were inoculated in triple samples either with FP9 virus or with the F11L knockout virus in a moi of 0.05 pfu/cell. Die The triplicate samples were each harvested at different times following infection and titrated under agar. The error bars show the standard deviations between the triplicate samples.

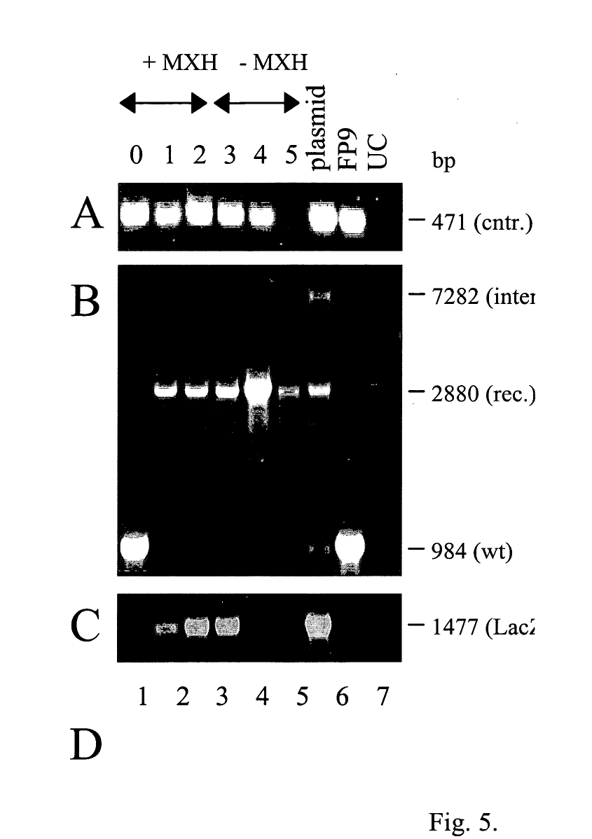

FIG. 5: PCR analysis of genomic DNA of the recombinant FWPV tyrosinase virus MT31. The initial plaque isolation (lane 0) and the first 2 subsequent plaque purification cycles (lanes 1 and 2) were carried out in the presence of selection medium (MXH) whereas the last 3 plaque purification steps (lanes 3 to 6) were carried out in the absence of MXH. pLGFV7.5-mTyr-DNA was used as control matrix DNA, FP9 is the wt virus control DNA and UC the uninfected control DNA. (A) Control PCR (F1-F2) showing the relative amount of virus DNA. (B) PCR F1-F4: The 984 bp band corresponds to the expected DNA fragment amplified from wt virus DNA (wt), the 7282 bp band corresponds to the amplification product containing the tyrosinase gene and the lacZ gpt subcassette contained in the intermediate recombinant virus (interm.), the 2880 bp band corresponds to the product which represents only the amplifications product of the tyrosinase gene expression cassette (rec.). (C) PCR PR43-44 showing the presence of the lacZ sequence. (D) Expression of the tyrosinase of mouse detected by the production of melanin in CEF. CEF cells in Petri dishes with 6 cm in diameter were infected with a moi of 0.1 pfu/cell. Six days following infection the cells were harvested, transferred into an U bottom microtiter plate and washed in PBS. Lanes 1-5: Cells infected with five different recombinant viruses; lane 6: uninfected cells; lane 7: cells infected with wt virus.

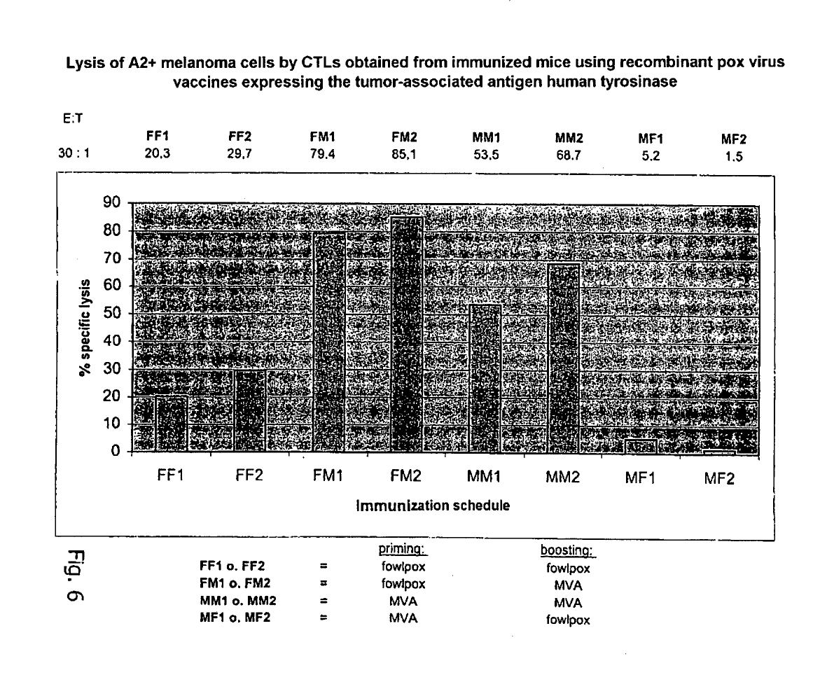

FIG. 6: Advantage of a combined vaccination with FWPV tyrosinase and MVA tyrosinase vaccines in the prime-boost method. Two mice per group were immunized in four week intervals twice each with 10⁸ infectious units of virus vaccine by intraperitoneal administration. The vaccinated groups were as follows:

Group FF: prime with FWPV tyrosinase and boost with FWPV tyrosinase

Group FM: prime with FWPV tyrosinase and boost with MVA tyrosinase

Group MM: prime with MVA tyrosinase and boost with MVA tyrosinase

Group MF: prime with MVA tyrosinase and boost with FWPV tyrosinase

Three weeks after the second immunization (boost) the tyrosinase-specific T cell response was examined in comparison. For this purpose, T cells from the spleen of the animals were prepared, cultured over a period of 7 days and then tested for their cytotoxic capacity for tyrosinase-specific target cells in the chromosome release test. Shown are the values obtained for each of the specific lyses of the target cells (in % at an effector/target ratio of 30:1). It was observed that the T cells of the animals which had received a combined vaccination in group FM clearly showed the highest reactivity. In contrast, in the mice of groups FF and MM which had received a homogenous immunization with respect to the vaccine only moderate cytotoxic responses could be measured. The lowest cytotoxicity was revealed in the test of the T cells of group MF which had been vaccinated first with the MVA tyrosinase and then with FWPV tyrosinase.

These results clearly support the superiority of a combined vaccination with FWPV tyrosinase vectors and MVA tyrosinase vectors as compared to the vaccination with each of the vectors vaccines alone. In this respect it seems to be of particular importance to use the FWPV vector vaccine as the primary vaccine.

Materials and Methods

Cells and Viruses

Primary chicken embryo fibroblasts (CEF) were prepared using 11 days old brooded eggs an cultured in MEM (Gibco) with 10% lactalbumin (Gibco) and 5% basal medium supplement (BMS—Seromed). HeLa cells and Vero cells were cultured in DMEM (Gibco) supplemented with 10% fetal calf serum (FCS) (Gibco). FWPV-FP9, a well characterized plaque isolate of attenuated strain HP1-438 (Boulanger et al., 1998) was cultured in the presence of MEM supplemented with 2% FCS on CEF.

Sequencing of Genomic FWPV DNA

FWPV-FP9 cultured on CEF were harvested following a freeze-thaw cycle. The virus was concentrated by ultracentrifugation and semi-purified through a 25% (w/w) sucrose cushion as described earlier (Boulanger et al., 1998). The pellet was resuspended in 0.05 M Tris, pH 8, with 1% SDS, 100 μM β-mercaptoethanol and 500 μg/ml of proteinase K and incubated for 1 hr at 50° C. The DNA was isolated following phenol/chloroform extraction, precipitated with ethanol and resuspended in H₂O. Sequencing was carried out by means of primer walking on the virus DNA. The first primer (PR30) was designed with respect to the partial sequence of the dove pox F11L gene published by Ogawa et al. (1993) under the accession number M88588. The primers used for sequencing were the following: PR30: 5′-CTCGTACCTTTAGTCGGATG-3′, PR31: 5′-GGTAGCTTTGATTACATAGCCG-3′, PR32: 5′-GATGGTCGTCTGTTATCGACTC-3′ und PR33: 5′-GTCTGATAGTGTATTAGCAGATGTAAAAC-3′.

Plasmid Constructions

(a) pBSLG. A lacZ gpt cassette of 4.2 bp corresponding to the cassette contained in plasmid pIIILZgpt described by Sutter & Moss (1992) and containing the E. coli lacZ gene under the control of the late vaccinia virus promoter P11 and the E. coli gpt gene under the control of the early/late vaccinia virus promoter P7.5 was directly inserted into the multiple cloning site of the pBluescript II SK+ plasmid (Stratagene) rendering plasmid pBSLG.

(b) pLGF11. The primers PRF1 (5′-GGCCGCGGCCGCCACTAGATGAACATGACACCGG-3′) and PRF2 (5′-GGCCCCCCGGGGCATTACGTGTTGTTTGTTGC-3′) containing a NotI and a SmaI restriction site (underlined), respectively, were used as a template for the amplification of the 471 base pairs (bp) long flank 1 sequence of the genomic virus DNA by means of PCR. This fragment was inserted into PBSLG which had been cleaved before with the same enzymes giving pBSLGF11. Flank 2 (534 bp) was amplified by using the primers PRF3 (5′-GGCCCCTGCAGGCAACAAACAACACGTAATGC-3′) and PRF4 (5′-CGCCCGTCGACCTTCTTTAGAGGAAATCGCTGC-3′) containing a PstI and a SalI restriction site (underlined), respectively. This fragment was inserted into pBSLGF11 digested previously with the two enzymes giving pLGF11.

(c) pIIIV7.5F11Rep and pLGFV7.5. The primers PRF5 (5′-GGCCCTACGTAGCAACAAACAACACGTAATGC-3′) and PRF6 (5′-GGCCGCGGCCGCCTCTATGTTTTTGTAGATATCTTTTTCC-3′) containing a Sna BI and a NotI restriction site (underlined), respectively, were used for the amplification of the 263 bp long sequence corresponding to a repeat at the 5′ end of flank 2 by means of PCR. This fragment was inserted upstream of the vaccinia virus P7.5 promoter sequence into plasmid pIIIdhrP7.5 (Staib et al., 2000) which had been digested previously with the same restriction enzymes. The flank 2-repeat-P7.5-promoter cassette was then excised from the plasmid thus obtained by means of digestion with PstI, treated with Klenow polymerase and inserted into the SmaI site of pLGF11 giving insertion plasmid pLGFV7.5.

(d) pLGFV7.5-mTyr. A unique PmeI site downstream of the vaccinia virus P7.5 promoter sequence in plasmid pLGFV7.5 was used to insert into this plasmid the gene coding for tyrosinase of mouse. Plasmid pZeoSV2+/muTy (Drexler et al., unpublished results) was digested by NheI and NotI. The desired fragment was treated with Klenow polymerase and inserted into the blunt end PmeI restriction site into pLGFV7.5 giving plasmid pLGFV7.5-mTyr.

Preparation of Mutant FWPV Virus

CEF infected by FWPV FP9 were transfected with plasmid pLGF11 using lipofectin (Gibco). The virus was harvested and plated under agar containing mycophenolic acid, xanthine and hypoxanthine (MHX-Medium). Viruses forming β-galactosidase-positive plaques were visualized using an XgaI coat and the plaques were purified twice in the presence of selection medium. LacZ/gpt+ viruses were further purified without selection medium until 100% blue plaques were obtained.

PCR Analysis of the Viral DNA

Total DNA was isolated from CEF infected with different selected virus isolates following treatment with proteinase K as described before (Boulanger et al., 1998) and analysed by means of PCR using the primers PRF1 and PRF4 to test for the presence of the wt sequence as well as primers PRF1 and PRF2 to test for the presence of DNA.

Analysis of Viral Growth

Confluent CEF were infected in triplicate with the wt virus or with the F11L mutant in a multiplicity of infection (moi) of 0.05 pfu/cell. The inoculate was removed 1 hr later and replaced by fresh medium. At different times following the infection, the flasks were removed from the incubator and stored at −80° C. The titer was determined after clearing the virus suspension at low speed by means of plaque test.

Preparation of Recombinant Virus

CEF infected with FWPV FP9 were transfected with linearized pLGFV7.5-mTyr plasmid DNA (FIG. 2). Recombinant viruses were purified three times in the presence of selection medium. For a new recombination to take place between flank 2 and the flank 2 repeat leading to a loss of the lacZ gpt subcassette, blue plaque isolates which had been propagated once on CEF were further purified in the absence of selection medium. Viruses forming white plaques were subsequently plaque-purified. The clones thus obtained were then tested by means of PCR as described before wherein additionally a PCR was carried out using the 2 primers (PR43: 5′-GACTACACAAATCAGCGATTTCC-3′ and PR44: 5′-CTTCTGACCTGCGGTCG-3′) specific for the lacZ sequence so that the presence of the selection cassette could be accessed.

Sequence Analysis of the F11L Gene

The FWPV-F11L gene is located in the central region of the virus genome (FIG. 1 B). Since the respective open reading frame in the genome of the CEF-adapted vaccinia virus strain MVA is fragmented (Antoine et al., 1998) we speculated that the gene probably might not be essential for FWPV replication. The partial sequence of the C terminus of the orthologous F11L gene of dove pox virus as well as the complete gene coding for the F12L dove pox virus orthologue and a partial sequence of the F13L orthologue were already known (Ogawa et al., 1993; accession number M88588). This published sequence overlaps a FWPV sequence comprising the complete F13L orthologue and almost the complete F12L orthologue (Calvert et al., 1992; accession number M88587). The two sequences overlap by 2598 bp and show 100% nucleotide identity. Assuming that the F11L orthologue is also highly conserved between dove pox and fowlpox viruses the first primer (PR30) used for sequencing of the FWPV gene was designed using the partial dove pox F11L sequence (453 bp) (FIG. 1A). The sequence obtained by using this primer (488 bp) exhibited 100% nucleotide identity with the end of the published dove pox sequence the following primers (PR31 to 33) were designed using the novel sequence to create overlaps which covered the F11L gene sequence twice (FIG. 1A). A sequencing was obtained for the last 1254 bp of the FWPV F11L ORF (FIG. 1A). A comparison with the published complete genomic sequence of FWPV (Afonso et al., 2000) revealed that this sequence is identical with the published sequence of ORF FPV110, the orthologous FWPV F11L gene.

Reading frame shifts of the F11L coding sequence in vaccinia virus MVA suggest that F11L probably is a non-essential gene which possibly could be used as an insertion site. Our analysis of the FWPV F11L protein (451 amino acids) using the GeneStream Align programme, however, reveals only 18,6% amino acid identity with the orthologue (354 amino acids) of the vaccinia virus strain Kopenhagen which could indicated different properties in both viruses. In the screening for possibly essential F11L gene functions, we found by means of BLAST no significant other homologies. Neither in the FWPV nor in the vaccinia virus protein were predicted any signal sequences or transmembrane domains.

Preparation of Viable FWPV Viruses with Mutant F11L

To determine whether FWPV-F11L can be used as a novel insertion site we constructed mutant viruses by means of insertion disruption of the coding F11L sequence. The plasmid pLGF11 containing the lacZ cassette flanked by 2 sequences of the FWPV F11L ORF (FIGS. 1B und 2) wa used for the preparation of recombinant viruses which were selected due to their growth in the presence of mycophenolic acid under an XgaI coat. The recombinants may be obtained either form a double recombination event both in flank 1 and flank 2 giving stable recombinant viruses, or by a single recombination event in one of the flanking gene sequences leading to unstable intermediate recombinant genomes. In the latter case further passages in the absence of selection medium are necessary which enable visualization of wt viruses as white plaques until a stable recombinant virus is obtained which only gives blue plaques. The genotype of successive virus isolates was characterized by means of PCR using the external primers which had been used for the generation of the flanks (PRF1 und PRF4). The presence of contaminating wt viruses was monitored by preferred amplification of the genomic wt sequence with respect to the shorter PCR product which rendered the test very sensitive. The viral clone F2 (FIG. 3A) had lost the wt gene sequence after 4 plaque purifications (clone F2.1.2.1.1). The viral clone F15 generating only blue plaques after 3 plaque purifications (F15.1.1.1) still contained the wt sequence as demonstrated by PCR (FIG. 3A). Following amplification of this viral clone (F15.1.1.1.1) by three successive passages CEF limited dilution also resulted in the presence of viruses giving rise to white plaques.

In an attempt to accelerate the isolation of recombinant viruses we tested the transfection with linearized plasmid DNA, a strategy recommended by Kerr & Smith (1991) to reduce the occurrence of single crossover events and the maintaining of plasmids derived from the resolution of unstable single crossover intermediates in viruses during vaccinia virus mutagenesis. The preparation of recombinant viruses using linearized plasmid should also be obtained due to a double recombination event and in the following directly lead to stable recombinant genomes. Indeed, viral clones F9, F10, and F16, prepared by linearized plasmid exhibited no detectable wt gene sequences even after the first plaque purification cycle (FIG. 3B). The viral clone F8 required only one more plaque purification to be obviously free from wt virus (FIG. 3B). Furthermore, plaque titration of F9.1.1.1.1 after three propagation cycles in CEF showed no more presence of contaminating wt virus.

Efficient In Vitro Culture of Virus with Mutant F11L

The successful generation of viruses with mutant F11L suggests that the F11L gene sequence is dispensable. To determine whether an inactivation can interfere with virus growth the mutant viral clone F9.1.1.1 was propagated and tested for multistep growth in CEF in comparison to wt FWPV (FIG. 4). Both viruses showed almost identical replications kinetics and generated equal amounts of infectious progeny.

F11L as a Target for Insertion Enables Stable Expression of Recombinant Genes

Because it was established that the F11L is non-essential and a disruption of the gene does not interfere with viral growth, the F11L gene locus was considered to be a suitable insertion site. Plasmid pLGF11 was used for the construction of a plasmid vector (pLGFV7.5) in order to be able to insert into the FWPV genome foreign genes together with the lacZ gpt selection subcassette under the control of the vaccinia virus P7.5 promoter (FIG. 2). The plasmid additionally contained a repeat of the flank 2 sequence (FIG. 2) in order to be able to remove the subcassette subsequently from the recombinant viruses. As the first foreign gene obtained by pLGFV7.5 the DNA sequence encoding the enzyme tyrosinase was inserted which is of interest as an antigen for an experimental vaccination against melanomas (Drexler et al., 1999). Tyrosinase is involved in the biosynthesis pathway of melanin. Cells expressing this enzyme accumulate melanin and become dark. This property provides a simple method for screening with respect to the expression of tyrosinase and the functional integrity thereof. Following transfection with pLGFV7.5-mTyr five recombinant viral clones were selected for further analysis. Linearization of the plasmid DNA which had proven to be very efficient during production of viruses with mutated F11L was used also for the preparation of recombinant viruses. The viral clones MT22 (data not shown) and MT31 (FIG. 5) showed no detectable wt virus sequence after only one plaque purification in the presence of selection medium (MT31.1, Spur 1, FIG. 5B). On this stage the genomic DNA preparation of both viral clones already revealed the presence of recombinant virus genomes which no longer contained detectable marker gene sequences (2880 bp gene product in FIG. 5B) and simultaneously contained the selected lacZ gpt-positive genotype (7282 bp) which is hardly detectable by this PCR reaction (see clone 31.1.1, FIG. 5B, third lane) but which is detected by PR43-44-PCR (FIG. 5C). From viral DNA of the fourth plaque purification of both clones, i.e. after only one plaque purification in the absence of selection medium, no marker sequence could be amplified (FIG. 5C). Following CEF infection all five recombinant viruses produce functional tyrosinase (FIG. 5D).

In addition, also the specific synthesis of melanin in infected HeLa and Vero cells was demonstrated which are both non-permissive for FWPV. The amount of melanin produced in these mammalian cells seemed to be smaller compared to the CEF infection. This could be either due to a lack of virus replication or a decreased expression of the tyrosinase gene or a less efficient melanin synthetic pathway in these cells (data not shown). To access the genetic stability of the tyrosinase insertion, all five recombinant virus isolates were amplified in four successive multistep growth passages on CEF and the virus progeny was analysed by means of plaque titration under agar. Of each of the recombinant viruses, ten different plaque isolates were examined for tyrosinase expression. Melanin synthesis was detected in all samples showing that each virus still generated functional recombinant enzyme (Table 1). The same test was carried out after six passages. Only one plaque isolate of one of the five recombinant viruses was no longer able to produce a functional tyrosinase (Table 1). PCR analysis of the virus DNA demonstrates that the genome of this virus clone probably still contained the recombinant full length gene sequences. This suggests that it is highly probable that the tyrosinase gene expression was inactivated by (a) point mutation(s) (data not shown).

The vaccinia virus F11L ORF potentially codes for a protein which has no homology or no characteristic motif which could predict a specific function. Therefore, the F11L orthologue of FWPV possibly is non-essential. In the present invention, this hypothesis was tested by insertion of a selection cassette into the FWPV-Gen containing a marker gene (lacZ) and a selectable gene (gpt). The generation of recombinant viruses containing this cassette and no longer wt gene sequences demonstrated that the orthologous full length FWPV gene is not essential for the growth of FWPV. The mutant virus grew as efficiently as the wt virus (FIG. 4) suggesting that the F11L gene locus can be considered as a suitable insertion site for recombinant genes. Consequently, we used this site to successfully generate FWPV viruses stably expressing the melanoma model antigen tyrosinase.

The stable expression of marker or selection genes in recombinant viruses can be unsuitable in the case of a use as a vector vaccine or for further genetic engineering. In our FWPV plasmid vector the selection subcassette was flanked by repeating sequences so that it could be eliminated afterwards. The preparation of such a recombinant first requires the isolation of a recombinant virus which contains only the selection subcassette but no longer wt sequence, and afterwards the isolation of the stable recombinant which has lost the selection subcassette. Therefore, the efficiency of the isolation strategy is important for the recovery of final recombinants within a reasonable amount of time. Similar to earlier studies (Leong et al., 1994; Sutter & Moss, 1992) we found that the combination of a reporter gene and a selectable gene is a simple and very efficient method of selection. This strategy was further improved by transfecting with linearized plasmid DNA. The recombination between the plasmid DNA and the virus genome can occur either by means of single crossover leading to integration of the complete plasmid sequence into the virus genome (Spyropoulos et al., 1988; Falkner & Moss, 1990; Nazarian & Dhawale, 1991) or can take place by double recombination. According to Spyropoulos et al. (1988) the frequency of both events is similar. In our hands the number of plaque purifications necessary to eliminate any wt sequence was indeed strongly reduced if linearized plasmid DNA was used (FIGS. 3 and 5). This technique not only allowed by a save of time but also lowered the risk of integrating random mutations into the virus genome which unavoidably occur during a number of passages. As suggested by Nazarian & Dhawale (1991) the total efficiency of recombination following transfection with linearized plasmid could be lower as if circular plasmid was used. However, in our hands the use of linearized plasmid did not reduce the efficiency of recombination since in the preparation of viruses with mutant F11L we obtained a ratio of one blue plaque following transfection with circular plasmid to five blue plaques following transfection with linearized plasmid. Furthermore, we obtained ratios between 1 and 10 in the preparation of other recombinant viruses (unpublished). Thus, our results confirm previous data (Spyropoulos et al., 1982) suggesting that the total frequency of recombination in vaccinia virus is not remarkably changed if the formation of single crossover recombinants is impeded by linearization of the plasmid in non-homologous regions.

An important aspect in the development of suitable virus vector vaccines is the stability of the recombinant viruses which can be fundamentally determined by the insertion site sought. The locus of the viral tyrosinekinase gene seems to be unsuitable for the preparation of recombinant avian pox viruses although it is the standard insertion site for the preparation of recombinant vaccinia virus (Scheiflinger et al., 1997; Amano et al., 1999). The stability of tyrosinase-recombinant FWPV viruses which may be obtained using F11L as the target can be easily monitored by the examination of melanin synthesis, simply examining the colour of the cell pellets (FIG. 5D and Table 1). After six passages on CEF only one plaque isolate of 50 did not express a functional recombinant gene indicating a high level of genomic stability.

Table 1: Stability of the murine expression of tyrosinase in 5 recombinant viruses

| | Number of isolates expressing | | | | |
|---|---|---|---|---|---|
| | murine tyrosinase among 10 plaques of each recombinant | | | | |
| | MT22.2.1.3. | MT22.2.1.4. | MT22.2.1.5. | MT31.1.1.1. | MT31.1.1.4. |
| n* | 1.1 | 1.1 | 1.1 | 1.1 | 1.1 |
| 4 | 10 | 10 | 10 | 10 | 10 |
| 6 | 10 | 9 | 10 | 10 | 10 |

*n= Number of passages in CEF with low multiplicity of infection

### REFERENCES

Afonso, C. L., Tulman, E. R., Lu, Z., Zsak, L., Kutish, G. F., Rock, D. L., 2000. The genome of pox virus. J. Virol. 74, 3815-31.
Amano, H., Morikawa, S., Shimizu, H., Shoji, I., Kurosawa, D., Matsuura, Y., Miyamura, T., Ueda, Y., 1999. Identification of the canarypox virus thymidine kinase gene and insertion of foreign genes. Virology 256,280-90.
Antoine, G., Scheiflinger, F., Domer, F., Falkner, F. G., 1998. The complete genomic sequence of the modified vaccinia Ankara strain: comparison with other orthopoxviruses. Virology 244, 365-96.
Belshe, R. B., Stevens, C., Gorse, G. J., Buchbinder, S., Weinhold, K., Sheppard, H., Stablein, D., Self, S., McNamara, J., Frey, S., Flores, J., Excler, J. L., Klein, M., Habib, R. E., Duliege, A. M., Harro, C., Corey, L., Keefer, M., Mulligan, M., Wright, P., Celum, C., Judson, F., Mayer, K., McKirnan, D., Marmor, M., Woody, G., 2001. Safety and immunogenicity of a canarypox-vectored human immunodeficiency virus Type 1 vaccine with or without gp120: a phase 2 study in higher-and lower-risk volunteers. J. Infect. Dis. 183, 1343-52.
Berencsi, K., Gyulai, Z., Gonczol, E., Pincus, S., Cox, W. L, Michelson, S., Kari, L., Meric, C., Cadoz, M., Zahradnik, J., Starr, S., Plotkin, S., 2001. A canarypox vector-expressing cytomegalovirus (CMV) phosphoprotein 65 induces long-lasting cytotoxic T cell responses in human CMV-seronegative subjects. J. Infect. Dis. 183, 1171-9.
Boulanger, D., Green, P., Smith, T., Czerny, C. P., Skinner, M. A., 1998. The 131-amino-acid repeat region of the essential 39-kilodalton core protein of fowlpox virus FP9, equivalent to vaccinia virus A4L protein, is nonessential and highly immunogenic. J Tirol 72,170-9.
Boursnell, M. E., Green, P. F., Campbell, J. L, Deuter, A., Peters, R. W., Tomley, F. M., Samson, A. C., Chambers, P., Emmerson, P. T., Binns, M. M., 1990. Insertion of the fusion gene from Newcastle disease virus into a non-essential region in the terminal repeats of fowlpox virus and demonstration of protective immunity induced by the recombinant. J. Gen. Virol. 71,621-8.
Boyle, D. B., Coupar, B. E., 1988. Construction of recombinant fowlpox viruses as vectors for poultry vaccines. Virus Res. 10, 343-56.
Cadoz, M., Strady, A., Meignier, B., Taylor, J., Tartaglia, J., Paoletti, E., Plotkin, S., 1992. Immunization with canarypox virus expressing rabies glycoprotein. Lancet 339, 1429-1432.
Calvert, J. G., Ogawa, R:, Yanagida, N., Nazerian, K., 1992. Identification and functional analysis of the fowlpox virus homolog of the vaccinia virus p37K major envelope antigen gene. Virology 191, 783-92.
Drexler, L, Antunes, E., Schmitz, M., Wolfel, T., Huber, C., Erfle, V., Rieber, P., Theobald, M., Sutter, G., 1999. Modified vaccinia virus Ankara for delivery of human tyrosinase as melanoma-associated antigen: induction of tyrosinase-and melanoma-specific human leukocyte antigen A*0201-restricted cytotoxic T cells in vitro and in vivo. Cancer Res. 59, 4955-63.
Falkner, F. G., Moss, B., 1990. Transient dominant selection of recombinant vaccinia viruses. J. Virol. 64, 3108-11.
Hodge, J. W., Sabzevari, H., Yafal, A. G., Gritz, L., Lorenz, M. G., Schlom, J., 1999. A triad of costimulatory molecules synergize to amplify T-cell activation. Cancer Res. 59, 5800-7.
Jenkins, S., Gritz, L., Fedor, C. H., Oneill, E. M., Cohen, L. K., Panicali, D. L., 1991. Formation of lentivirus particles by mammalian-cells infected with recombinant fowlpox virus. AIDS Res. Hum. Retrov. 7,991-998.
Kerr, S. M., Smith, G. L., 1991. Vaccinia virus-DNA ligase is nonessential for virus-replication-Recovery of plasmids from virus-infected cells. Virology 180,625-632.
Leong, K. H., Ramsay, A. J., Boyle, D. B., Ramshaw, 1. A., 1994. Selective induction of immune responses by cytokines coexpressed in recombinant fowlpox virus. J. Virol. 68, 8125-30.
Marshall, J. L., Hawkins, M. J., Tsang, K. Y., Richmond, E., Pedicano, J. E., Zhu, M. Z., Schlom, J., 1999. Phase I study in cancer patients of a replication-defective avipox recombinant vaccine that expresses human carcinoembryonic antigen. J. Clin. Oncol. 17, 332-7.
Moss, B., 1996. Genetically engineered poxviruses for recombinant gene expression, vaccination, and safety. Proc. Natl. Acad. Sci. USA 93, 11341-8.
Nazerian, K., Dhawale, S., 1991. Structural analysis of unstable intermediate and stable forms of recombinant fowlpox virus. J. Gen. Virol. 72, 2791-2795.
Ogawa, R., Calvert, J. G., Yanagida, N., Nazerian, K., 1993. Insertional inactivation of a fowlpox virus homolog of the vaccinia virus-F12L gene inhibits the release of enveloped virions. J. Gen. Virol. 74, 55-64.
Paoletti, E., 1996. Applications of pox virus vectors to vaccination: an update. Proc. Natl. Acad. Sci. USA 93, 11349-53.
Perkus, M. E., Tartaglia, J., Paoletti, E.,-1995. Poxvirus-based vaccine candidates for cancer, Aids, and other infectious-diseases. J. Leukocyte Biol. 58, 1-13.
Roth, J., Dittmer, D., Rea, D., Tartaglia, J., Paoletti, E., Levine, A. J., 1996. p53 as a target for cancer vaccines recombinant canarypox virus vectors expressing p53 protect mice against lethal tumor cell challenge. Proc. Natl. Acad. Sci: USA 93, 4781-6.
Scheiflinger, F., Falkner, F. G., Domer, F., 1997. Role of the fowlpox virus thymidine kinase gene for the growth of FPV recombinants in cell culture. Arch. Virol. 142, 2421-31.
Somogyi, P., Frazier, J., Skinner, M. A., 1993. Fowlpox Virus host-range restriction-Gene-expression, DNA-replication, and morphogenesis in nonpermissive mammalian-cells. Virology 197, 439-444.
Spehner, D., Drillien, R., Lecocq, J. P., 1990. Construction of pox virus vectors with intergenic insertions-Expression of the beta-galactosidase gene and the measles-virus fusion gene. J. Virol. 64,527-533.
Spyropoulos, D. D., Roberts, B. E., Panicali, D. L., and Cohen, L. K., 1988. Delineation of the viral products of recombination in vaccinia virus-infected cells. J. Virol. 62, 1046-1054.
Staib, C., Drexler, L, Ohlmann, M., Wintersperger, S., Erfle, V., Sutter, G., 2000. Transient host range selection for genetic engineering of modified vaccinia virus Ankara. Biotechniques 28, 1137-42, 1144-6, 1148.
Sutter, G., Moss, B., 1992. Nonreplicating vaccinia vector efficiently expresses recombinant genes. Proc. Natl. Acad. Sci. USA 89, 10847-10851.
Taylor, J., Weinberg, R., Languet, B., Desmettre, P., Paoletti, E., 1988. Recombinant fowlpox virus inducing protective immunity in non-avian species. Vaccine 6,497-503.
Wang, M., Bronte, V., Chen, P. W., Gritz, L., Panicali, D., Rosenberg, S. A., Restifo, N. P., 1995. Active immunotherapy of cancer with a nonreplicating recombinant fowlpox virus encoding a model tumor-associated antigen. J. Immunol. 154, 4685-4692.

1. A recombinant fowlpox virus (FWPV) containing at least one insertion of a foreign DNA in the F11L gene.

2. A recombinant fowlpox virus (FWPV) according to claim 1 wherein said foreign DNA has at least one foreign gene optionally in combination with a sequence for the regulation of the expression of the foreign gene.

3. A recombinant fowlpox virus (FWPV) according to claim 1 wherein said foreign DNA includes a regulatory sequence, preferably a pox virus-specific promoter.

4. A recombinant fowlpox virus (FWPV) according to claim 1 wherein said foreign gene codes for a polypeptide which preferably is therapeutically useful and/or codes for a detectable marker and/or is a selectable gene.

5. A recombinant fowlpox virus (FWPV) according to claim 4 wherein said therapeutically useful polypeptide is a component of a viral, bacterial, or parasitic pathogen or a tumor cell.

6. A recombinant fowlpox virus (FWPV) according to claim 5 wherein said therapeutically useful polypeptide is a component of HIV, Mycobacterium spp. or Plasmodium falciparum.

7. A recombinant fowlpox virus (FWPV) according to claim 5 wherein said therapeutically useful polypeptide is a component of a melanoma cell.

8. A recombinant fowlpox virus (FWPV) according to claim 1 wherein said detectable marker is a beta-galactosidase, beta-glucuronidase, a guanine ribosyl transferase, a luciferase, or a green fluorescent protein.

9. A recombinant fowlpox virus (FWPV) according to claim 8 wherein said marker gene and/or selectable gene can be eliminated.

10. A recombinant fowlpox virus (FWPV) according to claim 1 wherein the genomic region defined by nucleotide positions 131.860-131.870 in the fowlpox virus genome is the preferred site of integration in the F11L gene homologue.

11. A DNA vector containing a recombinant fowlpox virus (FWPV) according to claim 1 or functional parts thereof containing at least one insertion of a foreign DNA into the F11L gene, further preferred a replicon for the replication of the vector in a pro- or eukaryotic cell and a selection gene or a marker gene which is selectable in pro- or eukaryotic cells.

12. A pharmaceutical composition containing a recombinant fowlpox virus (FWPV) according to claim 1 or a DNA vector according to claim 11 in combination with pharmaceutically acceptable auxiliary agents and/or carries.

13. A pharmaceutical composition according to claim 12 in the form of a vaccine.

14. The use of a recombinant fowlpox virus, a DNA vector, or a pharmaceutical composition according to claim 1, claim 11, or claim 13 for the treatment of infectious diseases or tumor diseases.

15. A method for the preparation of a recombinant fowlpox virus or a DNA vector according to claim 1 or claim 11 wherein foreign DNA is introduced in the F11L gene of a fowlpox virus by recombinant DNA techniques.

16. The method according to claim 15 wherein the introduction is performed by homologous recombination of the viral DNA with the foreign DNA containing F11L-specific sequences, followed by propagation and isolation of the recombinant virus or the DNA vector.

17. A eukaryotic cell or prokaryotic cell containing a recombinant DNA vector or a recombinant virus according to claim 1.

18. A prokaryotic cell according to claim 17 which is a bacterial cell, preferably an E. coli cell.

19. A eukaryotic cell according to claim 18 which is a yeast cell, avian cell, preferably chicken cell, or a cell derived from a mammal, preferably a human cell.

20. A method for the immunization of a mammal, preferably a human, comprising the following steps:
a) priming of a mammal with a therapeutically effective amount of a fowlpox virus according to claim 1, a DNA vector according to claim 11 or a pharmaceutical composition according to claim 12,
b) optionally repeating said step a) between one and three times after between one week and eight months; and
c) boosting of the mammal with a therapeutically effective amount of another viral vector containing the same foreign DNA as the fowlpox virus, DNA vector or pharmaceutical composition in a).

21. The method according to claim 20 wherein the priming steps are carried out twice prior to boosting.

22. The method according to claim 21 wherein the priming steps are carried out at the beginning of the treatment and in week three to five, preferably week four of the immunization, wherein the boosting step is carried out in week eleven to thirteen, preferably week twelve of the immunization.

23. The method according to claim 20 wherein as the other viral vectors recombinant MVA, other avirulent vaccinia viruses and pox virus vectors, preferably recombinant forms of the vaccinia viruses NYVAC, CV-I-78, LC16m0, or LC16 m8, recombinant parapox viruses, preferably the attenuated Orf virus D1701, adenoviruses, preferably human adenovirus 5, orthomyxoviruses, preferably influenza viruses, herpes viruses, preferably human or equine herpes viruses, or alpha viruses, preferably Semliki Forest viruses, Sindbis viruses, or equine encephalitis viruses (-VEE) are used.

24. A combined preparation comprising the following components:
a) a fowlpox virus according to claim 1, a DNA vector according to claim 11, or a pharmaceutical composition according to claim 12, and
b) another viral vector containing the same foreign DNA as the fowlpox virus or the DNA vector of a).

25. A combined preparation according to claim 24 wherein as the other viral vectors recombinant MVA, other avirulent vaccinia viruses and pox virus vectors, preferably recombinant forms of the vaccinia viruses NYVAC, CV-I-78, LC16m0, or LC16 m8, recombinant parapox viruses, preferably the attenuated Orf virus D1701, adenoviruses, preferably human adenovirus 5, orthomyxoviruses, preferably influenza viruses, herpes viruses, preferably human or equine herpes viruses, or alpha viruses, preferably Semliki Forest viruses, Sindbis viruses, or equine encephalitis viruses (-VEE) are used.
